(19) United States
(12) Patent Application Publication
(10) Pub. No.: US 2006/0057169 A1
(43) Pub. Date: Mar. 16, 2006

(54) Topical anhydrous delivery system for antioxidants

(75) Inventors: Ratan Chaudhuri, LINCOLN PARK, NJ (US); Philip Linz, Cronton-on-Hudson, NY (US)

Correspondence Address: MILLEN, WHITE, ZELANO & BRANIGAN, P.C., 2200 CLARENDON BLVD., SUITE 1400, ARLINGTON, VA, 22201, US

(73) Assignee: MERCK PATENT GMBH, DARMSTADT (DE)

(21) Appl. No.: 10/534,034

(22) PCT Filed: Oct. 24, 2003
(86) PCT No.: PCT/EP03/11846

§ 371 Date: May 6, 2005

Related U.S. Application Data
(63) Continuation-in-part of application No. 10/616,494, filed on Jul. 10, 2003.
(60) Provisional application No. 60/424,316, filed on Nov. 7, 2002.
(60) Provisional application No. 60/424,316, filed on Nov. 7, 2002.
(60) Provisional application No. 60/395,612, filed on Jul. 15, 2002.

(51) Int. Cl. A61K 8/97

(52) U.S. Cl. 424/401

(57) ABSTRACT

This invention relates to an anhydrous composition comprising an antioxidant comprising over 40% by weight of hydrolysable tannins having molecular-weight of less than 1,000 and a substantially anhydrous or non-aqueous liquid vehicle functioning to disperse the antioxidant. The composition is suitable as cosmetic composition and/or therapeutic and/or prophylactic composition and/or anhydrous delivery system of cosmetic and/or pharmaceutical ingredients. The invention further relates to processes for producing such compositions.

### FIELD OF INVENTION

This invention relates to novel compositions including but not limited to cosmetic compositions and/or therapeutic and/or prophylactic novel anhydrous delivery systems of cosmetic and/or pharmaceutical ingredients, and especially those including low molecular-weight hydrolysable tannins (<1,000) found in extracts of Phyllanthus emblica (hereinafter PE extracts), and processes for producing such compositions.

As described in U.S. Pat. Nos. 6,124,268, 6,290,996 and 6,362,167 incorporated by reference herein, PE extracts provide significant skin care benefits, including, for example, skin-lightening or whitening effects and/or anti-oxidant effects and/or skin appearance regulating effects. The present invention is applicable to all types of extracts of Phyllanthus emblica. For example, in French patent 2730408 published Aug. 14, 1996, compositions are disclosed which are prepared by merely pressing the fruit or obtaining a dilute-alcoholic extract. Both the extracts obtained by pressing and the extracts obtained by alcoholic maceration may then be concentrated at a moderate temperature under reduced pressure, preferably less than 50° C., then optionally brought to the dry state by freeze-drying or any other method under reduced pressure and at a temperature that is lower than 50° C. so as to avoid degrading the active ingredients of the fruit. In greater detail, examples 3, 6 and 8 of the French patent 2730408 illustrate the manufacture and uses of extracts based on Phyllantus emblica.

In this French patent, however, there is no indication of the composition of the extracts. Conversely, in U.S. Pat. No. 6,124,268, Ghosal, issued Sep. 26, 2000 entitled “Natural Oxidant Compositions, Method For Obtaining Same And Cosmetic, Pharmaceutical and Nutritional Formulations Thereof” there is set forth the chemical composition of extracts of Emblica officinalis obtained by extracting the fresh fruit at elevated temperatures, e.g. 70° C., using a very dilute aqueous or alcoholic-water salt solution, e.g. 0.1 to 5%. By this extraction process, in the presence of sodium chloride, for example, hydrolysis of the glycocidic enzymes in the plant is prevented and the product is protected from microbial infestation.

In the Ghosal patent, the antioxidant blend of the constituents is described under the name of “CAPROS”, with claim 8, for example, of the patent setting forth the composition as follows:

An antioxidant blend consisting essentially of, by weight, (1) and (2) about 35-55% of the gallic/ellagic acid derivatives of 2-keto-glucono-δ-lactone; (3) about 4-15% of 2,3-di-O-galloyl-4, 6-(S)-hexahydroxydiphenoylgluconic acid; (4) about 10-20% of 2,3,4,6-bis-(S)-hexahydroxydiphenoyl-D-glucose; (5) about 5-15% of 3′,4′,5,7-tetrahydroxyflavone-3-O-rhamnoglucoside; and (6) about 10-30% of tannoids of gallic/ellagic acid.

The common names of the enumerated compounds are (1) and (2) Emblicanin A and Emblicanin B, (3) Punigluconin, (4) Pedunculagin and (5) Rutin.

A preferred antioxidant composition used in the present invention comprises a modification of the CAPROS composition, comprising a standardized extract of low molecular weight (<1000) hydrolyzable tannins, over 40%, preferably 50-80% w/w of Emblicanin A, Emblicanin B, Pedunculagin, and Punigluconin with low levels (<1%, w/w) of total flavonoids whereby the resultant products of the invention can be made into elegant white to off-white formulations. Such a composition is discussed with greater specificity in pages 28-30 of the August 2001 issue of Soap, Perfumery and Cosmetics, the article having the title Ingredients/Emblica, Bearing Fruit, by Ratan K. Chaudhuri. In the article that there is no mention, however, of any flavonoids much less the maximum acceptable amounts in the composition.

According to the preferred antioxidant composition, the total flavonoids are maintained at a level which does not impair the desired color, e.g. generally, by weight, less than about 1.0%, preferably less than about 0.8%, and even more preferably less than about 0.6%. Also, the desired concentrations of the Rutin species of flavonoids (3′,4′,5′,7-tetrahydroxyflavone-3-O-rhamnoglucoside) in the standardized extract are less than 1.0%, less than 0.01%, less than 0.001% and less than 0.0001%, with a value of 0.01 to 0.001% being particularly preferred. The most preferred concentrations of the components are on a percent by weight basis of the total dried extract:

| | Most Preferred Concentrations |
|---|---|
| | % by weight |
| Emblicanin A | 20-35 |
| Emblicanin B | 10-20 |
| Pedunculagin | 15-30 |
| Punigluconin | 3-12 |
| Total Flavonoids | <1 |

The standardized composition may exhibit average percentage deviations from these preferred values of:

| | | Most Preferred |
|---|---|---|
| | Preferred Deviation | Deviation |
| Emblicanin A | ±10% | ±5% |
| Emblicanin B | ±10% | ±5% |
| Pedunculagin | ±10% | ±5% |
| Punigluconin | ±10% | ±5% |
| Total Flavonoids | ±10% | ±5% |

The antioxidant composition can be obtained by removal of the total flavonoids by reversed-phase column chromatography or HPLC using a solvent system of acetonitrile, water/phosphoric acid (20/80/1) or other solvent combinations as they elute faster than the low molecular-weight tannins. Also, by selection of geographical location, the Phyllanthus emblica fruit extract may provide a substantially lower level of the total flavonoids (<1.0%). It has been obser/ed that medium-sized fruits collected from some parts of eastern India, during October-November, after water extraction and drying, yielded the preferred antioxidant composition as a powder with the desired low content of total flavonoids. Accordingly, by analyzing the total flavonoids content of extracts and selecting such extracts that contain the desired low content of total flavonoids, it is possible to prepare a standardized extract.

In the context of the present invention “flavonoids” include a family of compounds which exhibit a peak at 350 nm when analyzed by a UV spectrometer. Examples of flavonoids include but are not limited to flavonols and flavones, a species thereof being Rutin as discussed above.

In a preferred embodiment of this invention a substantially water-soluble (over 95% by weight) extract of Phyllanthus Emblica comprising less than 5% by weight of polymeric tannins, with substantially no black specks and at high levels, e.g. over 75% by weight of bio-active, low molecular-weight hydrolysable tannins having molecular weights below 1,000 is used. This extract can be obtained by an process which removes ologomeric and polymeric tannins. A suitable process comprises the following steps: 1) providing an extract of Phyllanthus Emblica either resulting from the original extract from the plant, or from a suspension of a powdered composition obtained after the extract is processed, e.g. after a drying step; 2) If necessary, physically separating the black specks and/or precursors thereof and/or polymeric tannins from the water-soluble components, for example by filtration with the use of a filter aid; 3) If desired, concentrating the resultant aqueous solution of the enriched composition of Emblica officinalis, for example to a dry powder.

Aqueous formulations of PE extracts are described in the above-identified patents and applications, these formulations being generally made by introducing a minor amount of powdered PE extracts into an aqueous solution along with known excipients. Whereas these formulations are commercially acceptable, a discoloration of such solutions has been observed after prolonged storage. The cause of such discoloration is believed to be due to the fact that the PE extracts contain polyphenolic compounds which are susceptible to aerial oxidation on the one hand and hydrolysis on the other hand; however, Applicants do not wish to be bound by this explanation of the mechanism of discoloration.

One aspect of the present invention therefore is to provide a delivery system which will inhibit or prevent discoloration, presumably by inhibiting or preventing such aerial oxidation and/or hydrolysis of active ingredients, PE extracts in particular. Other aspects of the invention are to provide a process for producing a final substantially anhydrous formulation and the resultant product. Still another aspect is a formulation that provides improved adhesion and skin-feel properties.

Upon further study of this application, other objectives and advantages of the invention will become apparent.

In order to attain certain objectives of the invention, formulations are provided which are not based on water, but instead are based on a substantially anhydrous or non-aqueous vehicle so that the final formulation contains preferably less than 1% by weight of water. Aside from being non-aqueous, the vehicle must be capable of dispersing the PE extracts with adjuvants if necessary. It is also preferred that the non-aqueous vehicle have an emollient function as well. Classes of vehicles to be used in the present invention include but are not limited to silicone fluids, organic esters and glycols.

Examples of silicone vehicles include but are not limited to cyclomethicone, both the tetramer and the pentamer, hexamethyldisiloxane, phenyltrimethicone cross linked polymers of dimethicone and cyclomethicone (hereinafter “crosspolymer”) methylvinylsiloxanes, methylvinylsiloxane-dimethylsiloxane copolymers, dimethylvinylsiloxy-terminated dimethylpolysiloxanes, dimethylvinylsiloxy-terminated dimethylsiloxane-methylphenylsiloxane copolymers, dimethylvinylsiloxy-terminated dimethylsiloxane-diphenylsiloxane-methylvinylsiloxane copolymers, trimethylsiloxy-terminated dimethylsiloxane-methylvinylsiloxane copolymers, trimethylsiloxy-terminated dimethylsiloxane-methylphenylsiloxane-methylvinylsiloxane copolymers, dimethylvinylsiloxy-terminated methyl(3,3,3-trifluoropropyl)polysiloxanes, and dimethylvinylsiloxy-terminated dimethylsiloxane-methyl(3,3-trifluoropropyl)siloxane copolymers, as well as functional derivatives thereof.

There are, moreover, innumerable polyorganosiloxane oils that are described in the commercial and patent literature, and it is expected that still other silicone oils will be developed in the future; so it is contemplated that all silicone oils will be useful in the present invention. For a further description of possible silicone oils that can be used as vehicles, see the discussion of the use of silicones to disperse particulate vitamin C for use as a topical composition in U.S. Pat. No. 6,146,664, to Siddiqui issued Nov. 14, 2000, especially column 8, incorporated by reference herein. Also U.S. Pat. No. 6,475,500 to Vatter et al. is of interest since it describes different anhydrous skin treatments including a cross linked siloxane elastomer gel of a specific yield point and volatile siloxane inclusions of the elastomers.

As for the anhydrous organic esters that can be used as vehicles in the present invention, preferred are those which also have emollient properties. Examples of such esters include but are not limited to cetearyl octanoate, caprylic/capric triglyceride, octylhydroxysterate, PPG-2 myristyl ether propionate, tentaerythrityl tetracaprylate/caprate, tentaerythrityl tetraisosterate, natural and synthetic jojoba oils, cetyl acetate, and acetylated lanolin alcohol.

Examples of glycols, include but are not limited to mono- or poly-alkylene glycols are contemplated, a non-limiting example being propylene glycol.

Whereas it is preferred that the vehicle has emollient properties, it is not necessary to use a vehicle that is also an emollient since it is possible to add emollients to the mixture of the vehicle and EP extracts.

In the substantially anhydrous formulation, the content of the vehicle is sufficient, generally, about 20-80%, preferably 20-60% by weight of the completed formulation to achieve the desired dispersibility of the PE extracts. The content of PE extracts in the formulation is generally about from 0.05 to 10%, preferably 0.1-3% by weight, with the preferred minimum weight ratio of the content of the vehicle to the content of the PE extracts being about 20:3.

Another aspect of this invention concerns the preferred addition of at least one structural and/or gelling agent. Such structural/gelling agents can be combined with the EP extracts to form a mixture comprising the PE extract with the structural agent, and/or the gelling agent. Likewise, the structural/gelling agent can be combined with the substantially anhydrous vehicle in order to form corresponding mixtures which thereafter can be combined with the PE extracts.

The structural agent which provides firmness, structure, consistency and thermal stability to the product can be selected from subgeneric classes of materials which include but are not limited to natural, modified or unmodified waxes, mineral waxes, high melting point fatty alcohols, glycerol or glycol esters, polyethylene and polyethylene glycol polymers.

Examples of the natural modified or unmodified waxes include but are not limited to beeswax, candelilla wax, carnauba wax, and hydrogenated castor wax.

Examples of mineral waxes include but are not limited to ozokerite and ceresin.

Examples of high melting fatty alcohols include but are not limited to cetyl alcohol and stearyl alcohol.

Examples of glycerol or glycol esters include but are not limited to Croda Syncrowaxes, i.e. 18-36 glycol esters.

An example of polyethylene glycol polymers includes but is not limited to Carbowax Sentry 1000.

The structural agents are incorporated in the final formulation at a level of about 5-50% by weight.

As for gelling agents which are also used in an amount of 5-50% by weight of the final formulation, subgeneric classes include but are not limited to silicone elastomers, gelled natural and mineral oil systems and gelled mineral oil and polymer systems.

Preferred examples of silicone elastomers include but are not limited to cyclomethicone and dimethicone cross polymers e.g. Dow Corning 9040, polysilicone-11 mixtures, e.g. Gransil PM Gel, and Gransil DCM, and Gransil DMIG-6.

Preferred examples of gelled natural and mineral oil systems include but are not limited to a mixture of canola oil and silica and corn starch, e.g. Vegelatum Clear; a mixture of canola oil, soy bean germ extract, corn starch and silica, e.g. Vegelatum Equiline; gelled castor oil and rice bran oil (Natunola Health).

Preferred examples of gelled mineral oil and polymer systems include but are not limited to esters of hydrogenated polyisobutene, ethylene/propylene/styrene copolymers, and butylene/ethylene/styrene copolymers, e.g. Versagel M, ME, MC, MD, ME, MJ and MP (Penreco Corp.); and polybutene, e.g. Indopol H-100.

The total amount of the sum of the structural agent and gelling agent will be determined by the desired rheological properties of the final formulation. As a guideline, the total amount of the sum in the final formulations will be in the range 5-90% by weight.

The substantially anhydrous delivery system of the present invention can be utilized for the incorporation of any PE-extract; however, the delivery system is particularly beneficial for the incorporation of the standardized extract described above and especially the commercial product EMBLICA™. It is also contemplated that the anhydrous delivery system of the present invention can be utilized for the incorporation of other active materials.

Additional ingredients can be added to the formulation for their known functions, for example skin lightening agents, skin brightening agents, skin even-toning agents, anti-aging agents, sunscreen agents, and antiperspirant/deodorant agents, herbal products, vitamins, and medicaments. Since rheological properties of the final product will primarily be dependent on the nature and proportion of the vehicle and the structural and gelling agents of same, the formulator can tailor make the final formulation to the desired product, e.g. semi-solid or gel.

Examples of antiperspirant agents include but are not limited to aluminum zirconium tetrachlorohydrex GLY (coordination complex of aluminium zirconium tetrachlorohydrate and glycerine)

Examples of additives for skin feel and adhesion include but are not limited to bismuth oxychloride, Boron Nitride, PPG-3 myristyl ether, glyceryl laurate, PEG-40 castor oil and PEG-derivatives of fatty alcohols and mixtures thereof. With respect to bismuth oxychloride in particular, it has been discovered that by the addition of same, important advantageous properties are imparted to the composition. Thus, the appearance and consistency of the final product may be improved considerably by the addition of generally about 0.5 to 20%, preferably 2 to 10% by weight, of powdered bismuth oxychloride pigment (e.g., Birono LF-2000). The formulated product with the pigment is whiter, has a more substantial appearance, and offers a much drier, silkier skin feel, than the same product without this pigment. Adhesion to the skin is also improved with the addition of this pigment. A list of some presently commercially available bismuth oxychlorides is given below.

| Commercial | | Particle Size in μm |
|---|---|---|
| Bismuth | Distinguishing | (Laserbeam Diffraction; |
| Oxychloride | Feature | Malvern 2000) |
| Biron ® B50 | Moderately heavy | 2.0-35.0 (80% within range) |
| | powder | 9.0-15.0 (D50: median size) |
| Biron ® Fines | Slightly less heavy, | 2.0-35.0 (80% within range) |
| | fine powder | 9.0-15.0 (D50: median size) |
| Mibiron ® | 50% mica | <50 (80% within range) |
| N50 | | 16.0-22.0 (D50: median range) |
| Biron ® NLD | Surfactant-treated, | 2.0-25.0 (80% within range) |
| | improved dispersibility | 6.0-12.0 (D50: median range) |
| Biron ® ESQ | Matte, less hiding, | 2.0-35.0 (80% within range) |
| | excellent skin adhesion | 11.0-17.0 (D50: median range) |
| Biron ® | Light-stable, excellent | <35.0 (80% within range) |
| LF-2000 | skin-feel | 8.0-20.0 (D50: median size) |
| Biron ® | Dispersed in | BiOCl (68.0-72.0%) |
| Liquid Silver | Octylhydroxy stearate, | Ethyhexyl Hydroxystearate |
| | Highly lustrous | (28.0-32.0%) |
| Biron ® MTU | Matte, transparent & | 2.0-35.0 (80% within range) |
| | light-stable | 12.0-18.0 (D50: median size) |

Iridescent bismuth oxychloride coated mica pigments are also contemplated. Whereas all bismuth oxychloride pigments will provide advantageous properties, the preferred pigments are Biron® LF-2000 and Biron® MTU.

Biron® LF-2000 is a white pigment having particle size (determined by Laserbeam Diffraction; Malvern 2000)<35 μm (80% within range) and 8.0-20.0 μm (D50: median size) which offers excellent skin feel, and adds some luster to the final product, and Biron® MTU is a white pigment having particle size 2.0-35.0 μm (80% within range) and 12.0-18.0 μm (D50: median range) which also offers improved skin feel and a more matte look to the product on the skin.

Other sources of Bismuth oxychloride can also be included in the invention, such as, a range of Bismuth oxychloride products available from Engelhard. These are:

| | |
|---|---|
| Pearlite ® 01 UVS | Some light-stability, lustrous |
| Pearlite ® 02 UVS | Some light-stability, matte |
| Pearlite ® 03 | Lustrous, poor UV stability |
| Pearlite ® 04 | Matte, poor UV stability |
| Mearlite ® GBU | Matte, poor UV stability |
| Mearlite ® LBU | Lustrous, poor UV stability |
| Mearlite ® GLS | Some light-stability, lustrous |
| Pearl-Glo ® UVR | Some light-stability, lustrous |

The advantages of the addition of bismuth oxychloride will benefit other anhydrous compositions having different or no anti-oxidants, i.e. compositions without PE. Such other compositions include color cosmetic products such as lipsticks, lip glosses, and lip balms. Anhydrous cream-to-powder foundations, creamy eye shadow, and blushers may also benefit from such a delivery system. Hair pomades, shaping balms, and molding waxes may also be formulated with this delivery system, as can various anhydrous ointment systems for such applications as diaper rash, muscle aches, and burns. It is also to be understood that PE, because of its antioxidant, anti-aging, skin lightening and skin even toning properties will impart improved properties to all of the products.

Other antioxidants may be incorporated in the system which include mixtures of antioxidants suitable for use in the cosmetic formulations. Known and commercial mixtures are, for example, mixtures comprising, as active ingredients, lecithin, L-(+)-ascorbyl palminate and citric acid (e.g. Oxynex® AP), natural tocopherols, L-(+)-ascorbyl palmitate, L-(+)-ascorbic acid and citric acid (e.g. Oxynex® K LIQUID), tocopherol extracts from natural sources, L-(+)-ascorbyl palmitate, L-(+)-ascorbic acid and citric acid (e.g. Oxynex® L LIQUID), DL-α-tocopherol, L-(+)-ascorbyl palmitate, citric acid and lecithin (e.g. Oxynex® LM) or butylhydroxytoluene (BHT), L-(+)-ascorbyl palmitate and citric acid (erg. Oxynex® 2004).

The formulations according to the invention can comprise vitamins as further ingredients. Preferably, vitamins and vitamin derivatives chosen from vitamin A, vitamin A propionate, vitamin A palmitate, vitamin A acetate, retinol, vitamin B, thiamine chloride hydrochloride (vitamin B1), riboflavin (vitamin B2) nicotinamide, vitamin C (ascorbic acid), vitamin D, ergocalciferol (vitamin D2), vitamin E, DL-tocopherol, tocopherol E acetage, tocopherol hydrogen-succinate, vitamin K1, esculin (vitamin P active ingredient), thiamine (vitamin B1) nicotinic acid (niacin), pyridoxine, pyridoxal, pyridoaxmine, (vitamin B6), panthothenic acid, biotin, folic acid and cobalamine (vitamin B12) are present in the cosmetic formulations according to the invention, particularly preferably vitamin A palmitate, vitamin C, DL-tocopherol, tocopherol E acetate, nicotinic acid, panthothenic acid and biotin.

Compositions of the present invention may also comprise one or more organic sunscreens. Suitable sunscreens can have UVA absorbing properties, UVB absorbing properties or a mixture thereof. The exact amount of the sunscreen active will vary depending upon the desired sun protection factor, i.e. the “SPF” of the composition as well as the desired level of UVA protection. The compositions of the present invention preferably comprise an SPF of at least 10, preferably at least 15. (SPF is a commonly used measure of photoprotection of a sunscreen against erythema. The SPF is defined as a ratio of the ultraviolet energy required to produce minimal erythema on protected skin to that required to products the same minimal erythema on unprotected skin in the same individual. See Federal Register, 43, No 166, pp. 38206-38269, Aug. 25, 1978). Compositions of the present invention preferably comprise from about 2% to about 25%, more typically from about 4% to about 15%, by weight, of organic sunscreen. Suitable sunscreens include, but are not limited to, those found in the CTFA International Cosmetic Ingredient Dictionary and H handbook, 7.sup.th edition, volume 2 pp. 1672, edited by Wenninger and McEwen (The Cosmetic, Toiletry, and Fragrance Association, Inc., Washington, D.C., 1997).

The compositions of the present invention preferably comprise a UVA absorbing sunscreen actives that absorb UV radiation having a wavelength of from about 320 nm to about 400 nm. Suitable UVA absorbing sunscreen actives are selected from dibenzoylmethane derivatives, anthranilate derivatives such as methylanthranilate and homomethyl, 1-N-acetylanthranilate, and mixtures thereof. Examples of dibenzoylmethane sunscreen actives are described in U.S. Pat. No. 4,387,089; and in Sunscreens: Development, Evaluation, and Regulatory Aspects, Second edition, edited by N. J. Lowe and N. A. Shaath, Marcel Dekker, Inc. (199,). The UVA absorbing sunscreen active is preferably present in an amount to provide broad-spectrum UVA protection either independently, or in combination with, other UV protective actives that may be present in the composition.

Preferred UVA sunscreen actives are dibenzoylmethane sunscreen actives and their derivatives. They include, but are not limited to, those selected from 2-methyldibenzoylmethane, 4-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′-diisopropylbenzoylmethane, 4-(1,1 -dimethylethyl)-4′-methoxydibenzoylmethane, 2-methyl-5-isopropyl-4′-methoxydibenzoylmethane, 2-methyl-5-tert-butyl-4′-methoxy-dibenzoylmethane, 2,4-dimethyl-4′-methoxydibenzoylmethane, 2,6-dimethyl-4′-tert-butyl-4′methoxydibenzoylmethane, and mixtures thereof. Preferred dibenzoyl sunscreen actives include those selected from 4-(1,1-dimethylethyl)-4′-methoxydibenzoylmethane, 4-isopropyldibenzoylmethane, and mixtures thereof.

A more preferred sunscreen active is 4-(1,1-dimethylethyl)-4′-methoxydibenzoylmethane also known as butyl methoxydibenzoylmethane or Avobenzone.

The compositions of the present invention preferably further comprise a UVB sunscreen active that absorbs UV radiation having a wavelength of from about 290 nm to about 320 nm. The compositions preferably comprise an amount of the UVB sunscreen active that is safe and effective to provide UVB protection either independently, or in combination with, other UV protective actives that may be present in the compositions. The compositions preferably comprise from about 1% to about 15%, more preferably from about 1% to about 12%, of UVB absorbing organic sunscreen.

A wide variety of UVB sunscreen actives are suitable for use herein. A list of currently approved sunscreens can be found in Organic Sunscreens published by R. Chaudhuri et al. in The Chemistry and Manufacture of Cosmetics, Vol. III, pages 627-644 (2002). Preferred UVB sunscreen actives are 3 selected from 2-ethylhexyl-2-cyano-3, 3-diphenylacrylate (referred to as octocrylene), Homomenthyl salicylate, 2-phenyl-benzimidazole-5-sulphonic acid (PBSA), cinnamates and their derivatives such as 2-ethylhexyl-p-methoxycinnamate and octyl-p-methoxycinnamate, TEA salicylate, octyldimethyl PABA, camphor derivatives and their derivatives, and mixtures thereof. Salt and acid neutralized forms of the acidic sunscreens are also useful herein. When organic sunscreen salts, such as PBSA, are used within compositions of the present invention they can disrupt the action of the thickener with the result that the final product may have sub optimal rheology. This can be countered by the addition of higher levels of thickener, fatty alcohols or nonionic surfactants such that the rheology of the final product returns to the desired level.

An agent may also be added to any of the compositions useful in the present invention to stabilize the UVA sunscreen to prevent it from photo-degrading on exposure to UV radiation and thereby maintaining its UVA protection efficacy. Wide ranges of compounds have been cited as providing these stabilizing properties and should be chosen to compliment both the UVA sunscreen and the composition as a whole. Suitable stabilizing agents include, but are not limited to, those described in U.S. Pat. Nos. 5,972,316; 5,968,485; 5,935,556; 5,827,508 and Patent WO 00/06110. Preferred examples of stabilizing agents for use in the present invention include 2-ethylhexyl-2-cyano-3,3-diphenylacrylate (referred to as octocrylene), ethyl-2-cyano-3,3-diphenylacrylate, 2-ethylhexyl-3,3-diphenylacrylate, ethyl-3,3-bis(4-methoxyphenyl)acrylate, and mixtures thereof. Di-2′-ethylhexyl-3,5-dimethoxy-4-hydroxy benzylidene malonate and other derivatives as exemplified in U.S. Ser. No. 09/904,904 filed Jul. 16, 2001 and Ser. No. 10/022, 343 filed Dec. 20, 2001, and published International Application No. PCT/EP 02/06743.

To summarize the quantitative amounts described above, the final formulation on a weight basis comprises in general about 20-80%, preferably 20-60% of a substantially anhydroLs liquid vehicie, a total of about 5 to about 90% of a structural and/or gelling agent, and 0.05-10%, preferably 0.1-3% of a PE extract, especially the extract having the trademark EMBLICA. Other components, especially bismuth oxychloride, can be incorporated in percentages that function for their intended purpose. The preferred combination of ingredients is 30-40% silicone oils (such as, 36.6% cyclomethicone), 50-70% structural or gelling agents (such as, 9.0% beeswax, 5.0% ozokerite, 45.4% Dow Corning 9040 Silicone Elastomer, 3.0%), 1-5% Bismuth oxychloride (such as, 3.0% Biron® LF-2000), and 0.1 to 5.0% PE extract (such as, 1.0% Emblica).

In order to produce the desired delivery system and the final formulation, there are several alternative processes which can be utilized. For example it is contemplated that all the desired components can be blended together under sufficient heat and mixing in a single step in order to form the final formulation. An improved process, however, involves more than one step.

Based on 100 parts by weight of the final formulation, the first step comprises blending a mixture of about 5 to 80% of a vehicle and 5 to 90 of a structural or gelling agent with sufficient heat, e.g. a temperature of about 60 to 90° C. and mixing until a clear and uniform mixture is obtained.

In a separate step the PE extract is mixed with a minor amount of, e.g. 1 to 20% of the same vehicle used in the first step together with a minor amount 1 to 30% of a structural and/or gelling agent. This subsequent step is important insofar as the mixture should be blended with sufficient heat but preferably below 60° C. until it is relatively smooth and contains no visible lumps. The product from this subsequent step is then mixed with that of the first step containing the major amounts of vehicle and structural/gelling agents, the mixing being conducted at preferably below 60° C., for example 40-50° C. so as to avoid any decomposition of the PE extract. By virtue of this two step operation, the ingredients in the first step can be heated to a higher temperature which will facilitate mixing, and the resultant mixture then can be cooled to below 60° C. before mixing with the minor composition containing the PE extract and the minor amounts of vehicle and structural/gelling agents.

Optionally, other components can be added: for example after the first step, an antiperspirant agent can be added to the product of the first step and then blended therein. In such a process, the subsequent step mentioned above, would be a third step after the antiperspirant agent is blended and the resultant mixture is cooled to below 60° C.

Final product can be packaged in any suitable container for daily use for multiple applications. Also, this product can be provided as a single dose application, for example in gelatin capsules.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius; and, unless otherwise indicated, all parts and percentages are by weight.

### EXAMPLE 1

### Anhydrous Delivery System for Emblica™ without bismuth Oxychloride

| INCI NAME | TRADE NAME/MANUFACTURER | % |
|---|---|---|
| Phase A | | |
| Beeswax | White Beeswax SP-422/Strahl & | 9.00 |
| | Pitsch | |
| Ozokerite | White Ozokerite SP-1020/Strahl & | 5.00 |
| | Pitsch | |
| Cyclomethicone | Dow Corning 345 Fluid/Dow Corning | 36.00 |
| Cyclomethicone (and) | Down Corning 9040 Silicone | 40.00 |
| Dimethicone | Elastomer Blend/Dow Corning | |
| Crosspolymer | | |
| Phase B | | |
| Cyclomethicone | Dow Corning 345 Fluid/Dow Corning | 3.60 |
| Cyclomethicone (and) | Dow Corning 9040 Silicone Elastomer | 5.40 |
| Dimethicone | Blend/Dow Corning | |
| Crosspolymer | | |
| Phyllanthus emblica | Emblica ™/RONA | 1.00 |
| fruit extract | | |
| Total | | 100.00 |

Procedure: Blend ingredients in Phase A; heat with mixing at about 70<80° C. until clear and uniform. Blend ingredients in Phase B separately at a temperature below 60° C., e.g. room temperature; the mixture should be smooth and contain no lumps. Cool Phase A to about 60° C. and add Phase B with mixing. When the mixture is uniform it may be packaged.

### EXAMPLE 1A

### With Bismuth Oxychloride

| INCI NAME | TRADE NAME/MANUFACTURER | % |
|---|---|---|
| Phase A | | |
| Beeswax | White Beeswax SP-422/Strahl & | 9.00 |
| | Pitsch | |
| Ozokerite | White Ozokerite SP-1020/Strahl & | 5.00 |
| | Pitsch | |
| Cyclomethicone | Dow Corning 345 Fluid/Dow Corning | 36.00 |
| Cyclomethicone (and) | Down Corning 9040 Silicone | 40.00 |
| Dimethicone | Elastomer Blend/Dow Corning | |
| Crosspolymer | | |
| Phase B | | |
| Bismuth Oxychloride | Biron ® LF-2000/Rona | 3.00 |
| Phase C | | |
| Cyclomethicone | Dow Corning 345 Fluid/Dow Corning | 3.60 |
| Cyclomethicone (and) | Dow Corning 9040 Silicone Elastomer | 5.40 |
| Dimethicone | Blend/Dow Corning | |
| Crosspolymer | | |
| Phyllanthus emblica | Emblica ™/RONA | 1.00 |
| fruit extract | | |
| Total | | 100.00 |

Procedure: Blend ingredients in Phase A; heat with mixing until clear and uniform. Blend bismuth oxychloride into Phase A. Blend ingredients in Phase C separately; the mixture should be smooth and contain no lumps. Cool Phase A to 60-65° C. and add Phase C with mixing. When the mixture is uniform it may be packaged.

The addition of bismuth oxychloride as the lustrous white powder Biron® LF-2000 whitens the gel and allows for greater skin adhesion and a much smoother, silkier skin feel to the final product. These benefits can also be obtained in other systems, as illustrated in Examples 3 and 4 below. Note that the viscosity of the final product may be varied, from a stable solid, as in Examples 1, 2 and 3, to a flowable gel, as in Example 4. In these formulas, as above, Biron® LF-2000 adds whiteness, a smoother skin feel, and greater skin adhesion to the final product.

### EXAMPLE 2

### Antiperspirant with Emblica™

| INCI NAME | TRADE NAME/MANUFACTURER | % |
|---|---|---|
| Phase A | | |
| Stearyl Alcohol | Crodacol S-70/Croda | 18.00 |
| Hydrogenated | Castor Wax/Ross | 5.00 |
| Castor Oil | | |
| Cyclomethicone | Dow Corning 345 Fluid/Dow Corning | 40.90 |
| PPG-3 Myristyl Ether | Varonic APM/Goldschmidt | 3.00 |
| Glyceryl Laurate | Jeechem MLD/Jeen | 4.00 |
| Phase B | | |
| Aluminum Zirconium | Rezal 36 GP Superfine/Reheis | 20.00 |
| Tetrachlorohydrex | | |
| GLY | | |
| Bismuth Oxychloride | Biron © MTU/RONA | 4.00 |
| Phase C | | |
| Cyclomethicone | Dow Corning 345 Fluid/Dow Corning | 1.80 |
| Cyclomethicone (and) | Dow Corning 9040 Silicone | 2.70 |
| Dimethicone | Elastomer Blend/Dow Corning | |
| Crosspolymer | | |
| Phyllanthus emblica | Emblica ™/RONA | 0.50 |
| Fruit Extract | | |
| Phase D | | |
| Fragrance | Grapefruit Fragrance 26520M/Shaw | 0.10 |
| | Mudge | |
| Total | | 100.00 |

Procedure: Blend ingredients in Phase A; heat with mixing at 70-80° C. until clear. Add Phase B with mixing. Blend ingredients in Phase C separately; the mixture should be smooth and contain no lumps. Cool the Phase A/B mixture to below 60° C., e.g. 40-50° C. and add Phase C with mixing. Add Phase D with mixing. Package.

### EXAMPLE 3

### Anhydrous Oil-Free Emblica™ Gel

| INCI NAME | TRADE NAME/MANUFACTURER | % |
|---|---|---|
| Phase A | | |
| Ozokerite | White Ozokerite SP-1020/Strahl & | 3.00 |
| | Pitsch | |
| Cyclomethicone | Dow Corning 345 Fluid/Dow Corning | 25.00 |
| Cyclomethicone (and) | Gransil GCM/Grant Industries | 60.00 |
| Polysilicone-11 | | |
| Phase B | | |
| Bismuth Oxychloride | Biron ® LF-2000/Rona | 2.00 |
| Phase C | | |
| Cyclomethicone | Dow Corning 345 Fluid/Dow Corning | 3.60 |
| Cyclomethicone (and) | Dow Corning 9040 Silicone Elastomer | 5.40 |
| Dimethicone | Blend/Dow Corning | |
| Crosspolymer | | |
| Phyllanthus emblica | Emblica ™/RONA | 1.00 |
| Fruit Extract | | |
| Total | | 100.00 |

Procedure: Blend ingredients in Phase A; heat with mixing until clear and uniform. Add bismuth oxychoride and disperse with mixing. Blend ingredients in Phase C separately; the mixture should be smooth and contain no lumps. Cool Phase A/B to 50-60° C. and add Phase C with mixing. When the mixture is uniform it may be packaged.

### EXAMPLES 4-9

### Additional Formulas with Phyllanthus emblica Extract

| | Formula Number | | | | | |
|---|---|---|---|---|---|---|
| Raw Material | 4 | 5 | 6 | 7 | 8 | 9 |
| PHASE A | | | | | | |
| Ozokerite | 2.80 | 5.00 | 6.00 | 5.00 | 3.00 | 3.00 |
| Dow Corning 345 | 48.50 | 30.00 | 50.00 | 25.00 | 27.00 | 25.00 |
| Dimethicone (and) | 38.70 | — | — | 60.00 | 60.00 | — |
| Polysilicone-11 (1) | | | | | | |
| Phenyl Trimethicone (and) | — | 55.00 | — | — | — | — |
| Polysilicone-11 (2) | | | | | | |
| Cyclomethicone (and) | — | — | 34.00 | — | — | — |
| Dimethicone Crosspolymer (3) | | | | | | |
| Cyclomethicone (and) | — | — | — | — | | 60.00 |
| Polysilicone-11 (4) | | | | | | |
| Bismuth Oxychloride (5) | — | — | — | — | — | 2.00 |
| PHASE B | | | | | | |
| Dow Corning 345 | 3.60 | 3.60 | 3.60 | 3.60 | 3.60 | 3.60 |
| Cyclomethicone (and) | 5.40 | 5.40 | 5.40 | 5.40 | 5.40 | 5.40 |
| Dimethicone Crosspolymer (3) | | | | | | |
| Phyllanthus emblicaextract | 1.00 | 1.00 | 1.00 | 1.00 | 1.00 | 1.00 |
| Total | 100.00 | 100.00 | 100.00 | 100.00 | 100.00 | 100.00 |

Procedure: Follow procedure as described in the Example 3
Note:
Trade Names
(1) Gransil DMG-6, Grant Industries
(2) Gransil PM Gel, Grant Industries
(3) Dow Corning 9040 Silicone Elastomer Blend, Dow Corning
(4) Gransil GCM, Grant Industries
(5) Biron LF-200, Rona

The preceding examples can be repeated with similar success by substituting the generically or specifically described reactants and/or operating conditions of this invention for those used in the preceding examples.

### EXAMPLE 10

### Anhydrous System with Sunscreens

| INCI NAME | TRADE NAME/MANUFACTURER | % |
|---|---|---|
| Phase A | | |
| Beeswax | White Beeswax Sp-422/Strahl & | 9.00 |
| | Pitsch | |
| Ozokerite | White Ozokerite SP-1020/Strahl & | 5.00 |
| | Pitsch | |
| Cyclomethicone | Dow Corning 345 Fluid/Dow | 36.00 |
| | Corning | |
| Cyclomethicone (and) | Dow Corning 9040 Silicone | 37.00 |
| Dimethicone | Elastomer Blend/Dow Corning | |
| Crosspolymer | | |
| Phase B | | |
| Bismuth Oxychloride | Biron ® LF-2000/Rona | 3.00 |
| Phase C | | |
| Ethylhexylmethoxy | Eusolex 2291/Rona | 6.00 |
| cinnamate | | |
| Avobenzone | Eusolex 9020/Rona | 2.00 |
| Di-ethylhexyl- | Oxynex ST/Rona | 2.00 |
| Syringylidene malonate | | |
| Total | | 100.00 |

Procedure: Blend ingredients in Phase A; heat with mixing until clear and uniform. Blend bismuth oxychloride into Phase A. Blend ingredients in Phase C separately; apply heat if needed. Cool Phase A to 60-65° C. and add Phase C with mixing. When the mixture is uniform it may be packaged.

### EXAMPLE 11

### Anhydrous System with Sunscreens

| INCI NAME | TRADE NAME/MANUFACTURER | % |
|---|---|---|
| Phase A | | |
| Beeswax | White Beeswax Sp-422/Strahl & | 9.00 |
| | Pitsch | |
| Ozokerite | White Ozokerite SP-1020/Strahl & | 5.00 |
| | Pitsch | |
| Cyclomethicone | Dow Corning 345 Fluid/Dow Corning | 36.00 |
| Cyclomethicone (and) | Dow Corning 9040 Silicone | 37.00 |
| Dimethicone | Elastomer Blend/Dow Corning | |
| Crosspolymer | | |
| Phase B | | |
| Bismuth Oxychloride | Biron ® LF-2000/Rona | 3.00 |
| Phase C | | |
| Homosalate | Eusolex HMS/Rona | 6.00 |
| Avobenzone | Eusolex 9020/Rona | 2.00 |
| Di-ethylhexyl-Syringal | Oxynex ST/Rona | 2.00 |
| malonate (proposed) | | |
| Total | | 100.00 |

Procedure: Blend ingredients in Phase A; heat with mixing until clear and uniform. Blend bismuth oxychloride into Phase A. Blend ingredients in Phase C separately; apply heat if needed. Cool Phase A to 60-65° C. and add Phase C with mixing. When the mixture is uniform it may be packaged.

The entire disclosure of all applications, patents and publications, cited above and below, including but not limited to U.S. patent application Ser. No. 10/120,156 filed Apr. 11, 2002 and Provisional application 60/395,612 filed Jul. 5, 2002 is hereby incorporated by reference.

From the foregoing description, one skilled in the art can easily ascertain the essential characteristics of this invention and, without departing from the spirit and scope thereof, can make various changes and modifications of the invention to adapt it to various usages and conditions.

1. An anhydrous composition comprising
(a) an antioxidant comprising over 40% by weight of hydrolysable tannins having a molecular-weight of less than 1,000.
(b) a substantially anhydrous or non-aqueous liquid vehicle functioning to disperse the antioxidant.

2. An anhydrous composition according to claim 1, wherein the antioxidant comprises Emblicanin A, Emblicanin B, Pedunculagin and Punigluconin, preferably in an amount of 40-80% by weight.

3. An anhydrous composition according to claim 1, wherein the antioxidant comprises by weight: 20-35% Emblicanin A, 10-20% Emblicanin B, 15-30% Pedunculagin-and 3-12% Punigluconin and preferably the antioxidant has a content of Rutin of less than 0.01% by weight and preferably of flavonoids in general of less than 0.01% by weight.

4. A composition according to claim 1, wherein the antioxidant has maximum absorbances (optical density) in the UV region of 0.8 at wavelength 410 nm, 0.1 at wavelength 470 nm, 0.08 at wavelength 530 nm, 0.09 at wavelength 590 nm, and 0.02 at wavelength 650 nm.

5. An anhydrous composition according to claim 1, wherein the substantially anhydrous or non-aqueous liquid comprises at least one member selected from the group consisting of silicone fluids, organic esters and glycols, wherein the composition comprises preferably at least one silicone fluid.

6. An anhydrous composition according to claim 1, wherein the composition further comprises at least one structural agent and wherein said structural agent is preferably selected from the group consisting of high melting point fatty alcohols, glycerol esters, glycol esters, polyethylene polymers and polyethylene glycol polymers.

7. An anhydrous composition according to claim 1, wherein the composition further comprises a gelling agent, wherein said gelling agent preferably comprises at least one member selected from the group consisting of silicone elastomers, gelled natural and mineral oil systems, and gelled mineral oil and polymer systems.

8. An anhydrous composition according to claim 1, wherein the composition further comprises at least one sunscreen.

9. An anhydrous composition according to claim 1, further comprising an amount of bismuth oxychloride sufficient to impart an improved skin feel to the composition, wherein the bismuth oxychloride is preferably included as a predispersion.

10. A method of producing an anhydrous composition according to claim 1, said anhydrous composition further comprising each one of a structural and gelling agent, said process comprising the steps of:
(1) mixing up to 80% of said substantially anhydrous or non-aqueous vehicle and 5 to 90% of a structural and/or gelling agent with sufficient heat and mixing until a clear and uniform mixture is obtained.
(2) mixing the anti-oxidant with a minor amount of about 1-20% of said substantially anhydrous or non-aqueous vehicle with a minor amount of about 1-30% of said structural and/or gelling agent, under a sufficient heat but below 60° C. until it contains no visible lumps, and
(3) mixing the product of step (2) with the product of step (1) at below 50° C.
